(19) United States
(12) Patent Application Publication
(10) Pub. No.: US 2004/0116513 A1
(43) Pub. Date: Jun. 17, 2004

(54) Method to alter the isomeric profile of trans fatty acid in ruminant meat and milk and to increase the concentration of $I(cis)-11 conjugated linoleic acid

(76) Inventors: Yvan Larondelle, Brussel (BE); Michel Focant, Brussel (BE); Eric Mignolet, Amay (BE); Juha Mikko Griinari, Espoo (FI)

Correspondence Address: KNOBBE MARTENS OLSON & BEAR LLP, 2040 MAIN STREET, FOURTEENTH FLOOR, IRVINE, CA, 92614, US

(21) Appl. No.: 10/450,632

(22) PCT Filed: Dec. 21, 2001
(86) PCT No.: PCT/EP01/15386

(30) Foreign Application Priority Data
Dec. 22, 2000 (EP) 00870324.1

(51) Int. Cl. A61K 31/355

(52) U.S. Cl. 514/458

(57) ABSTRACT

The present invention relates to methods for altering the fatty acid composition in milk or tissue fat directly derived from a milk producing ruminant The methods consist of administering to said milk producing ruminant a suitable amount of a chemical compound selected from the vitamin E family, or a structurally or functionally related compound or derivative thereof. At high doses, vitamin E influences ruminal biohydrogenation and allows the production of milk or tissue fat with a highly desirable fatty acid profile: high trans-11 C_{18:1}, high cis-9, trans-11 C_{18:02} (CLA), low trans-10 C_{18:1}. Methods are disclosed to obtain said desirable fatty acid profile, thereby improving the nutritional benefits to human health associated with CLA. Milk and tissue fat obtained by said methods are also disclosed. Dietary intakes of cis-9, trans-11 C_{18:2} CLA and trans-11 C_{18:1} fatty acids in milk or meat, or products thereof, produced in accordance with the present invention in ruminant animals, can be effective in preventing cancer in different sites, reduce risk of coronary heart disease and to enhance immune function.

### FIELD OF THE INVENTION

The present invention generally relates to a method of improving the quality of milk and meat products and to the derived products thereof. In a first aspect, it relates to a method for altering the fatty acid composition in milk or tissue fat directly derived from a ruminant. More particularly, the inventors pertain to a method of increasing the cis-9, trans-11 conjugated linoleic acid C_{18:2} (CLA) content and the level trans-11 C_{18:1} fatty acids in said milk or tissue fat. Milk and tissue fat containing said increased levels of CLA and trans fatty acids are also disclosed. In another aspect the invention relates also to a suitable feed for use in said method.

### BACKGROUND

Conjugated linoleic acid (CLA) refers to a collection of eighteen carbon fatty-acids with conjugated double bonds in variable positions and geometric configurations. Researchers first became interested in CLA as an anticarcinogenic factor found in ruminant fat (Pariza et al. 1985). Since the initial identification by Pariza and coworkers, the list of potential health effects demonstrated in animal models has been further extended and includes today antidiabetic, antiatherogenic, antiobesity, and immune stimulating effects (Pariza 1999). Milk fat typically contains one major isomer, cis-9, trans-11 CLA and several other minor isomers (Sehat et al. 1998; Bauman et al. 2000a).

The cis-9, trans-11 isomer possesses anticarcinogenic activity and is effective when consumed as a component of foods. The anticarcinogenic effect was demonstrated in a rat model using NMU (methylnitroso-urea) as the carcinogen (Ip et al. 1999). Intake of dairy products, in particular full fat milk, has been associated with decreased risk of breast cancer (Knekt et al. 1996). In another study examining the relationship between dietary intakes of CLA from natural foods and breast cancer risk in postmenopausal women, intake of cheese was found to be correlated with lower risk of breast cancer (Aro et al. 2000). Increased serum levels of cis-9, trans-11 CLA were also predictive of lower breast cancer risk in this study. Protective effect of whole milk against breast cancer was associated in these study populations with two to three fold variation in CLA intakes estimated to be from 320 to 700 mg/d in the study by Knekt et al. (1996) and 70 to 200 mg/d in the study by (Aro et al. 2000). If CLA will be confirmed to be an important contributing factor to the lower risk of breast cancer in human population, enhancement of the concentration of CLA in dairy products by two to three fold could have a major impact. In addition to its anticarcinogenic effect, the cis-9, trans-11 isomer of CLA may also stimulate the immune function and reduce the atherogenesis in the cardiovascular system.

Cis-9, trans-11 CLA is formed as an intermediate in the biohydrogenation of linoleic acid (cis-9, cis-12 C_{18:2}) in the rumen (Griinari & Bauman, 1999). A number of dietary situations that affect concentration of CLA in milk fat have been described (Bauman et al. 2000). Commercial products of CLA are produced by alkali isomerization of high linoleate oils and contain ideally a 1:1 mixture of two CLA isomers, cis-9, trans-11 and trans-10, cis-12 (Reaney et al. 1999). Trans-10 cis-12 CLA is present at low levels in milk fat (1% of total CLA) and it possesses a set of physiological effects that are distinct from the effects of the cis-9, trans-11 isomer (Pariza 1999). Enhancement of cis-9, trans-11 CLA in milk fat by natural dietary means is a cost-effective strategy to enrich our diets with a potentially health-promoting component.

Dietary fats of ruminant animal origin, in particular dairy products, are the main source of dietary CLA (Fritsche et al. 1999). This is wholly related to the presence of the rumen and rumen micro-organisms, which biohydrogenate polyunsaturated fatty acids of the diet (FIG. 1). During the process of biohydrogenation some of the fatty acid intermediates escape from the rumen and become absorbed in the small intestine, and subsequently incorporated into milk fat. These intermediates include cis-9, trans-11 CLA and trans-11 C_{18:1}, both of which contribute to milk fat cis-9, trans-11 CLA since trans-11 C_{18:1} can be desaturated back to cis-9, trans-11 C_{18:2} in the mammary gland (Griinari et al. 2000). This process involves the action of delta-9 desaturase, an enzyme active in a number of different tissues in particular in the cow's mammary gland. Thus, a feeding strategy that enhances the ruminal formation of trans-11 C_{18:1} will equally increase milk fat output of CLA. Example 1 demonstrates the feasibility of CLA enhancing strategy, which is based on dietary supplementation with fish oil. Feeding of fish oil results in altered rumen biohydrogenation, which increases formation of vaccenic acid in the rumen. Increase in ruminal outflow of vaccenic acid alone is sufficient to enhance concentration of CLA in milk fat.

A number of reviews, patents and patent applications disclosed strategies to enhance CLA levels in milk fat (Griinari and Bauman 1999; Bauman et al. 2000b; Chilliard et al. 2000). These feeding strategies typically involve the use of vegetable oils as a source of unsaturated fatty acids.

U.S. Pat. No. 5,770,247 discloses a method to increase the CLA content of milk which consists of feeding to a lactating cow a diet which contains about 1% to about 5% of vegetable oil containing linoleic or linolenic acid.

WO 99/63991 discloses a method to increase levels of trans-C_{18:1} fatty acids and in particular of the trans-11 isomer (vaccenic acid) in ruminants. These trans fatty acids might then be fed to other mammals and these mammals will form CLA from them. Said method involves feeding ruminants high amounts of linoleic acid, i.e. diets high in corn oil or soy oil which leads to high levels of trans-11 C_{18:1}, and/or lowering the pH of the rumen to allow for the accumulation of vaccenic acid, the substrate for cis-9, trans-11 CLA found in food products of production animals.

It is also known that supplementation of dairy cow diets with fish oils enhances milk fat CLA content. In this respect, WO 99/08540 discloses a dairy product having a fatty acid composition which includes at least 15 mg CLA/g of fat and preferably also includes at least about 10 mg omega-3 fatty acid/g of fat. Said dairy product is produced by a ruminant which has been fed a diet which includes a fish-derived product such as fish oil or fish meal. The diet typically includes about 0.5% to about 5% by dry weight of oil supplied by the fish-derived product.

The problem underlying the present invention is that in the methods described above and used to increase the CLA content, the final output of CLA is limited due to the deleterious effect of increased formation of trans-10 C_{18:1}. Dietary addition of vegetable or fish oils have the major disadvantage that they result in increased formation of trans-10 C_{18:1} on the expense of trans-11 C_{18:1} (Griinari et al, 1998, Griinari and Bauman, 1999). This results in decreased rates of CLA biosynthesis in the mammary gland.

Rumen bacteria are sensitive to the content of vegetable and fish oils in the diet, because unsaturated fatty acids contained in these oils have an unspecified toxic effect on the rumen bacteria. Biohydrogenation is thought to be the mechanism that aids rumen bacteria to deal with the presence of unsaturated fatty acid in their environment. However, when the load of unsaturated fatty acids in the diet exceeds biohydrogenation capacity, growth as well as function of rumen bacteria is impaired. Impairment of rumen bacterial function is consistent with decreased rates of fibre digestion in the rumen (Palmquist 1984) and decreased rate of milk fat synthesis in the mammary gland (Bauman and Griinari 2000) observed in conjunction with feeding high levels of vegetable and fish oils (a situation often referred to as milk fat depression). Change in rumen environment leading to decreased fibre digestion and reduced milk fat is associated with altered rumen biohydrogenatlon characterised by a shift in major biohydrogenation intermediates: decreased formation of trans-11 C_{18:1} and increased formation of trans-10 C_{18:1} in the rumen. In the remaining part of the text, the expression “trans-11 to trans-10 shift” is used to describe this process. This shift is also depicted in FIG. 1. As a result of the trans-11 to trans-10 shift, ruminal output of trans-10 C_{18:1} is increased and consequently the output of trans-11 C_{18:1} is decreased. Due to the importance of trans-11 C_{18:1} as the precursor of milk fat CLA, milk fat CLA content decreases (Bauman et at. 2000a). As demonstrated by the work of Bauman et al. (2000a), CLA content in milk fat can be increased up to ten fold, but it appears that this effect is transient due to the trans-11 to trans-10 shift in the rumen. In another example, cows fed with a diet that maximally increases milk fat content show a decrease in milk CLA content after the 8^{th} milking. This decrease is produced by the trans-11 to trans-10 shift and it is associated with reduced milk fat.

Therefore, it would be advantageous to have a method to prevent this trans-11 to trans-10 shift in the rumen and to maintain trans-11 C_{18:1} levels and subsequently the CLA-levels in milk fat. The present invention provides a method wherein the above stated problems are at least partially overcome by using a chemical compound selected from the vitamin E family, or a structurally related compound or derivative thereof, or another chemical compound with similar properties.

The method presented in this application will further enhance feeding strategies described in patent by U.S. Pat. No. 5,770,247, WO 99/08540 and WO 99/63991 to obtain higher levels of CLA enrichment in milk fat. The invention presents a complementary method. However, the method of the present invention can be used alone as such, or in combination and/or in any other feeding situations than those described in the prior art.

It is an objective of the present invention to provide a method to support the maintenance of high levels of CLA in milk or tissue fat directly derived from a ruminant.

In particular, it is an objective of the present invention to provide a method which allows to obtain high rates of trans-11 C_{18:1} formation in the rumen. As a result, said method allows to increase the amounts of trans-11 C_{18:1} available to the mammary gland or other tissues and increases levels of CLA in milk fat or tissue fat of said animal.

It is another objective of the present invention to increase trans-11 C_{18:1} formation in the rumen when animals are fed diets that otherwise would result in formation of large amounts of trans-10 C_{18:1}, for instance diets with high unsaturated fatty acid content and/or low effective fibre content and/or high starch content.

It is yet another objective to alter milk fat and tissue fat composition favourably by increasing the proportion of trans-11 C_{18:1} and cis-9, trans-11 CLA.

Another objective is to improve fibre digestion in the rumen and increase energy available to the ruminant animal.

Another objective is to improve milk production or growth when energy intake is limiting or to decrease feed consumption when energy intake is not limiting.

This invention can be applied to all domestic ruminants including bovine, ovine and caprine species plus others.

This invention can be applied on lactating as well as growing animals to alter fatty acid composition in milk and tissue lipids.

### DETAILED DESCRIPTION OF THE INVENTION

The present invention generally relates to a method of treating a ruminant. It relates to methods of improving the quality of milk and meat products produced by said ruminants, in particular by increasing the content of trans-11 C_{18:1} fatty acid and CLA of said products. In a first aspect the invention provides for a method for increasing the content of trans-11 C_{18:1} fatty acid and CLA in milk or tissue fat directly derived from a ruminant, comprising the step of administering to said ruminant a suitable amount of a chemical compound selected from the group consisting of the vitamin E family, comprising α-tocopherol, β-tocopherol, χ-tocopherol, δ-tocopherol, tocotrienols or a structurally or functionally related compound such as derivatives of tocopherols such as chroman analogs and trolox, synthetic phenol compounds, such as di-tert-butylhydroxy toluene (BHT), tert-butylhydroxy anisole (BHA), natural phenol compounds such as flavonoids, secondary antioxidants that support vitamin-E effect such as ascorbic acid or ubiqulnol or derivatives or mixtures thereof.

For purposes of definition throughout this description, it is understood herein that a fatty acid is an aliphatic (saturated or unsaturated) monocarboxylic acid. Lipids are understood to be fats or oils including the glyceride esters of fatty acids along with associated phosphatides, sterols and related compounds. A commonly employed shorthand system is used in this specification to denote the structure of the fatty acids. This system uses the letter “C” accompanied by a number denoting the number of carbons in the hydrocarbon chain, followed by a colon and a number indicating the number of double bonds, e.g. C20:5 eicosapentaenoic acid.

Also used in the description is “CLA” or “conjugated linoleic acid” which means a fatty acid that is an octadecadienoic acid having a conjugated double bond structure at any of several possible positions on the 18 carbon chain (7,9 to 12,14). The double bond at each of these positions can be in either the cis or trans configuration. The term CLA as used herein refers to a fatty acid composition made up of one or more of the “conjugated linoleic acid” isomers.

The term “fatty acid composition” means the identifiable fatty acid residues in the various triacylglycerols in the fat of said composition. In this description, the amount of an individual fatty acid is stated as mg of the fatty acid per gram of total fatty acids.

According to a first embodiment of the invention, a method for altering the content of trans-11 C_{18:1} fatty acid and CLA in milk or tissue fat directly derived from a ruminant includes administering to said ruminant a suitable amount of a chemical compound selected from the vitamin E family. Compounds of the vitamin-E family include, alpha-, beta-, gamma-, delta-tocopherols and -tocotrienols plus all their similar acting derivatives.

According to a further embodiment, active compounds might include not only the members of the vitamin E family but also a group of structurally or functionally (secondary antioxidants that potentiate vitamin-E effect, other free radical scavengers) related compounds or derivatives thereof.

Non limiting examples of structurally/functionally related compounds are given hereunder.

1. Derivatives of tocopherols (such as chroman analogs and trolox),

2. Synthetic phenol compounds [such as di-tert-butylhydroxy toluene (BHT), tert-butylhydroxy anisole (BHA)],

3. Natural phenol compounds (such as flavonoids),

4. Secondary antioxidants, such as compounds active in the aqueous phase such as ascorbic acid or compounds active in the lipid phase such as ubiquinol, and derivatives thereof.

Said compounds can be mixed with other feed, or can be used alone as a separate supplement. The amount of compound to be administered depends on the particular compound used.

Another aspect according to the invention is to provide a feed or a diet for ruminants resulting in increasing the content of trans-11 C_{18:1} fatty acid and CLA in milk or tissue fat directly derived there from. A suitable diet comprises administering more than 3 IU, preferably more than 6 IU vitamin E/kg body weight/day, yet more preferably about 15 to about 20 IU vitamin E/kg body weight/day.

Suitable feeds according to the invention An animal feed able to alter the trans-11 C_{18:1} fatty acid and CLA content in milk or tissue fat directly derived from a ruminant, comprising a suitable amount of a chemical compound selected from the group consisting of the vitamin E family, comprising α-tocopherol, β-tocopherol, χ-tocopherol, δ-tocopherol, tocotrienols or a structurally or functionally related compound such as derivatives of tocopherols such as chroman analogs and trolox, synthetic phenol compounds, such as di-tert-butyihydroxy toluene (BHT), tert-butylhydroxy anisole (BHA), natural phenol compounds such as flavonoids, secondary antioxidants that support vitamin-E effect such as ascorbic acid or ubiquinol or derivatives or mixtures thereof.

In an example a feed comprises vitamin E in an amount of more than 1500, preferably more than 3000 IU, yet more preferably more than 4000, more than 5000 or more than 6000 vitamin E/day.

This invention can be applied to all domestic ruminants including bovine, ovine and caprine species plus others, for example a cow, goat, sheep, buffalo, deer etc. This invention can be applied on lactating as well as growing animals to increase content of trans-11 C_{18:1} fatty acid and CLA in milk or tissue fat.

Milk and other animal products are essential food sources for a majority of the population. For instance, a problem in milk fat is the high level of medium chain saturated fatty acids (lauric (C_{12:0}), myristic (C_{14:0}) and palmitic acid (C_{16:0})) which have been associated with increased risk of cardiovascular disease. On the other hand, milk fat is relatively low in oleic acid (C_{18:1} cis) as well as in polyunsaturated fatty acids. The newest focus of interest is the conjugated linoleic acid, that is CLA, contained in milk and tissue fat, which has been shown to decrease the risk of cancer in many animal and in vitro tests. Also, vaccenic acid (trans-11 C_{18:1}) contained in milk fat is a desired component, because it has been shown to convert to CLA in humans under the effect of the delta-9-desaturase enzyme (Adlof et al., 2000).

Thus, it is desirable to produce milk and meat products with improved nutritional characteristics. The present invention accomplishes these goals by increasing the proportion of both trans-11 C18:1 and cis-9, trans-11 CLA in milk or tissue fat. This increase of CLA content gives the milk and meat product anti-carcinogenic activity and potentially other positive health benefits including enhancement of immune function and reduction in atherogenesis, as discussed in the background section. Enhancement of concentration of vaccenic acid in milk and meat possibly adds to the health benefits of dietary CLA given that vaccenic acid can be converted to cis9, trans-11 CLA in human tissues. The inventors surprisingly found that addition of vitamin E to feed of cows at higher levels than routinely added will minimise the formation of trans-10 C_{18:1} and support higher rates of trans-11 C_{18:1} formation in the rumen (example 2). In turn, higher rates of CLA synthesis are obtained in the mammary gland or other tissues, resulting in higher levels of CLA in milk or tissue fat.

Thus, a suitable amount of a member of the vitamin E family, or a structurally or functionally related compound or derivative thereof as claimed in the first embodiment, will influence biohydrogenation and allow the production of milk or tissue fat with a highly desirable fatty acid profile in terms of trans and conjugated fatty acids: high trans-11 C_{18:1,} high cis-9, trans-11 C_{18:2} and low trans-10 C_{18:1}. Furthermore, said member of the vitamin E family or related compound will avoid the shift from trans-11 to trans-10 biohydrogenation, which classically occurs when for instance cows are fed diets with high doses of vegetable oils or fish oils, or with products containing these oils.

A person skilled in the art could replace vitamin E (all-rac-α-tocopheryl acetate) by any other structurally or functionally related compound, which is able to produce the same reaction as claimed in the first embodiment.

According to a more preferred embodiment of this invention, said chemical compound is a member of the vitamin E family. According to another more preferred embodiment, said member of the vitamin E family is a-tocopherol in its esterified form (all-rac-α-tocopheryl acetate).

According to an even more preferred embodiment, vitamin E is administered in an amount of more than 2 preferably more than 6 IU/kg body weight/day. According to the invention, 1 International Unit (IU) of vitamin E corresponds to 1 mg of α-tocopheryl acetate.

Assuming a body weight of 500 kg for a cow, said amount of vitamin E as defined above corresponds to more than 1500 and preferably more than 300 IU vitamin E/day.

According to yet another more preferred embodiment, said suitable amount results in administering about 15 to about 20 IU vitamin E/kg body weight/day.

Again, assuming a body weight of 500 kg for a cow, said amount of vitamin E as defined above corresponds to about 7500 IU to about 10 000 IU vitamin E/day.

A comparable amount (IU/day) for other ruminants can easily be established. The daily dose for other ruminants can be determined experimentally, i.e. IU/kg of body weight/d can be used to provide the first estimate. The experimental protocol can be described as follows:

feed the animal a high concentrate low forage diet (diet consisting of 60 to 80% concentrate and 20 to 40% forage)

add 5% sunflower oil to the diet (other oils high in linoleic acid such as corn oil, safflower oil can also be used)

add 3 levels of vitamin-E starting from 3 up to 6 IU/kg body weigth/day

continue feeding for 2 weeks and take milk samples after the first and second weeks

analyse milk samples for fatty acid composition

recommended dose is the level that produces the highest trans-11 to trans-10 C_{18:1} ratio.

Vitamin E is widely used in dairy cows feeding to supplement low concentrations of vitamin E in the basal feeds. Feed with low vitamin E content are known from European patent 0429879B1. Current recommendation for use of vitamin E to prevent deficiency is to add enough vitamin E to reach a level of 15 IU/kg feed (INRA 1988). Typically this means an addition of 300 IU/d/cow. In addition to the basal dietary supplementation, vitamin E is added to lactating dairy cow diets to improve mammary gland health. Both scientists and manufacturers of vitamin E recommend the use of 1500 IU/d/cow (Smith et al. 2000; BASF AG. Technical Specification TS 9503). However, none of these publications discloses the addition of a high amount of vitamin E comprising at least more than 3000 IU vitamin E/day) to the feeds. Also, in none of these publications the addition of vitamin E aims to increase the content of trans-11 C_{18:1} and CLA in milk fat of cows.

As described further below, the inventors tested a dose of about 9600 IU vitamin E/day in cows. This amount was found to increase the content of trans-11 C18:1 and CLA in milk fat of said cows. However the effective dose might be lower but above the basal level of 1500 or preferably 3000 IU vitamin E/day.

Vitamin E has a toxic effect only at very high dosages. In growing chicks, daily intake of 1000 IU/kg of body weight is without negative effects. Corresponding dietary dose in a 500 kg lactating dairy cow would be 500 000 IU/d which far exceeds the effective dose range in this invention as described before.

The use of vitamin E as a feed supplement for altering the fatty acid composition of milk of cows is illustrated in example 2. In this experiment, 3 groups of cows were fed either a control diet, a diet supplemented with oilseed, and a diet supplemented with oilseed+9616 IU/d vitamin E. In terms of trans fatty acids, the values obtained in the milk clearly indicate that the oilseed diet induces a shift in the ruminal biohydrogenation process, resulting in formation of a proportionally higher amount of trans-10 C_{18:1} than of trans-11 C_{18:1}. This shift in major biohydrogenation intermediates was completely abolished by the supplementation of the oilseed diet with vitamin E. This vitamin E effect allows the rumen to produce more trans-11 C_{18:1}, which explains the increased content of trans-11 C_{18:1} and CLA in milk fat.

Vitamin-E is thought to support the growth and function of bacteria that produce trans-11 C_{18:1} as an intermediate, probably in two complementary ways: by acting in place of α-tocopherylquinol in the electron transfer chain which provides electrons for the reduction of cis-9, trans-11 C_{18:2} to trans-11 C_{18:1} and/or by inhibiting the growth and function of bacteria that produce trans-10 C_{18:1} as an intermediate i.e. the competing pathway. The competing may pathway produce trans-10, cis-12 C_{18:2} and subsequently trans-10 C_{18:1} in a process activated by free radical exchanges. Thus, vitamin-E may inhibit this process by scavenging the free radicals.

Bacteria that produce tran-11 C_{18:1} include many species of the fibre-digesting bacteria e.g. Butyrivibrio fibrisolvens. Thus, supporting the growth of the fibre-digesting bacteria may improve fibre digestion in the rumen and subsequently enhance the energy supply to the animal.

According to yet another embodiment, said chemical compound selected from the vitamin E family or structurally or functionally related compound or derivative thereof as defined above, is fed in admixture with a diet increasing the CLA content in milk or tissue fat.

More particularly, it can be fed in conjunction with diets intending to enhance ruminal formation of trans-11 C_{18;1}, and subsequently CLA content in milk fat. The composition of such diets is described in published reviews (Griinari and Bauman 1999; Bauman et al. 2000; Chilliard et al. 2000). For instance, it is known that vegetable oils or fish oils added to lactating dairy cow diets enhance the proportion of unsaturated fatty acids in milk fat, enhance the formation of trans-11 C_{18:1} in the rumen and subsequently the formation of CLA in the mammary gland. However, dietary addition of these oils result in increased formation of trans-10 C_{18:1} on the expense of trans-11 C_{18:1} (Griinari et al, 1998, Griinari and Bauman, 1999). This results in decreased rates of CLA biosynthesis in the mammary gland. The inventors found that vitamin E avoids this shift from trans-11 to trans-10 biohydrogenation, and allows to maintain high levels of trans-11 C_{18:1} in the rumen.

According to yet another embodiment, the ruminant in the method of the invention is a lactating ruminant.

According to a yet another more preferred embodiment, said lactating animal is a cow.

According to another embodiment, the present invention relates to a method as described above for increasing the CLA content in milk or tissue fat directly derived from a ruminant.

As mentioned before, there is interest in increasing milk or tissue fat content of CLA because it is one of the most potent, naturally occurring anti-carcinogens. Of the limited number of naturally occurring substances that have been demonstrated to have anti-carcinogenic activity in experimental models, all are of plant origin except for CLA. Indeed, cis-9, trans-11 C_{18:2} is a unique anti-carcinogenic agent because it is mainly present in foods from animal sources.

Experimental animal models have provided evidence that CLA can help to decrease the incidence of cancer and suggest that CLA intake during mammary gland development may provide lasting protection against subsequent tumorigenesis (Ip et al., 1994). CLA have also other positive biological effects including enhanced immune function and reduction of atherogenesis.

According to yet another embodiment, the present invention relates to a method as described above for increasing the trans-fatty acid 11 C_{18:1} content in milk or tissue fat directly derived from a milk producing ruminant.

Increasing the trans-fatty acid 11 C_{18:1} content in milk or tissue fat is desirable since vaccenic acid can be converted to CLA in human tissues (Adlof et al., 2000; Turpeinen et al., 2001). Vaccenic acid fed to animals also resulted in formation and tissue accumulation of cis-9, trans-11 CLA and CLA-metabolites which are thought to be the biologically active derivatives of dietary CLA (S. Banni, unpublished data).

According to yet another embodiment, the present invention relates to a method as described above for decreasing the trans-fatty acid 10 C_{18:1} content in milk or tissue fat directly derived from a ruminant.

Decreasing the trans-fatty acid 10 C_{18:1} content in milk or tissue fat is desirable since dietary intake of trans-10 C_{18:1}, determined as a concentration of this trans fatty acid isomer in platelet lipids, was positively associated with the degree of coronary artery disease (Hodgson et al., 1996).

According to yet another embodiment, the present invention relates to a method as described above for increasing the content of both trans 11 C_{18:1} fatty acid and CLA, without simultaneously increasing the trans 10 C_{18:1} fatty acid content in milk or tissue fat directly derived from a milk producing ruminant.

According to yet another embodiment, the present invention relates to a method as described above for preventing the trans-fatty acid 10 C_{18:1} formation in the rumen of a ruminant.

Formation of trans-10 C_{18:1} in the rumen of cows has been described in the literature (Griinari et al. 1998; Griinari & Bauman 1999). Increased formation of trans-10 C_{18:1} is known to be associated with decreased formation of trans-11 C_{18:1} in the rumen and with lower concentrations of CLA in milk fat (Griinari et al. 1999).

Before the discovery of the vitamin E effect according to this invention, the only method known in the art to prevent trans-10 C_{18:1} formation in the rumen was to add buffering agents to the diet (Piperova et al. 2000; WO 99/63991). Unfortunately, dietary buffers will also decrease overall production of trans-C_{18:1} fatty acids in the rumen and therefore this dietary strategy will not increase trans-11 C_{18:1} supply to the mammary gland and biosynthesis of CLA.

According to another embodiment, the present invention relates to the raw milk or tissue fat obtained by the method of the invention as described above.

As used in this invention, the terms raw milk and tissue fat refer to isolated milk or tissue fat in the form directly derived from a ruminant.

The method according to this invention allows to produce raw milk and tissue fat with a desirable fatty acid profile: high levels of high trans-11 C_{18:1} and cis-9, trans-11 C_{18:2} and low levels of trans-10 C_{18:1}.

According to a more preferred embodiment, the raw milk or tissue fat of the invention as described above has a fatty acid composition comprising at least 1% of the total fatty acids in the form of cis-9, trans-11 C_{18:2}.

According to yet another embodiment, the raw milk or tissue fat of the invention as described above has a fatty acid composition comprising at least 2% of the total fatty acids in the form of trans-11 C_{18:1}.

According to yet another embodiment, the raw milk or tissue fat of the invention as described above has a fatty acid composition characterized by a trans-11 C_{18:1}/trans-10 C_{18:1} ratio higher than 2.

As illustrated in example 2, by feeding cows a diet containing oilseed+9616 IU/d vitamin E, the obtained milk composition consisted of 9 mg trans-10 C_{18:1}/g of total fatty acids, 34 mg trans-11 C_{18:1}/g of total fatty acids of and 14 mg cis-9,trans-11 CLA/g of total fatty acids. For purposes of comparison, feeding a diet without supplementation of vitamin E resulted in a milk fat composition of 27 mg trans-10 C_{18:1}/g of total fatty acids, 25 mg trans-11 C_{18:1}/g of total fatty acids and 10 mg cis-9,trans-11 CLA/g of total fatty acids. It is clear that the above claimed levels can be achieved by the method according to the invention.

The raw milk and tissue fat of the invention can be used directly or as an additive to enhance the content of CLA and other beneficial unsaturated fatty acids of foods for human intake or for animal feed. The complex lipids containing CLA and other beneficial unsaturated fatty acids can also be extracted from the milk or tissue fat with solvents, and utilized in a more concentrated form (e.g. encapsulated) for pharmaceutical purposes and industrial application.

The raw milk and tissue fat of the invention can be used for preparation of food for preventing cancer and atherogenesis and to stimulate immune function. Food products containing a beneficial profile of CIA and other beneficial unsaturated fatty acids can be administered to humans or animals for the prevention of various diseases. It is here assumed that the intake of CLA can be effective for the prevention of various types of cancer, for reduction of coronary heart disease and for enhancement of immune function.

According to yet another embodiment, the present invention relates to a nutritional product comprising raw milk or tissue fat of the invention as described above, or a derivative thereof.

Said nutritional product means any food product comprising the milk or tissue fat of the invention in raw or processed form. For instance, said nutritional products can be a dairy product including the milk of the invention in raw or processed form. Examples of such dairy products are natural and processed cheeses, yoghurt, butter, ice cream, butter milk, cultured butter milk, cottage cheese, sour cream, frozen yoghurt, anhydrous butter oil, anhydrous butter fat, powdered milk, condensed milk, evaporated milk and other milk-based products. Also included in the invention are meat products comprising the fat of the invention in raw or processed form.

According to a further embodiment, the present invention also relates to an animal feed supplement suitable for use in the method according to the invention.

Said animal feed supplement can be mixed with a commercial feed or to a ration prepared on farm.

Said supplement comprises a chemical product selected from the vitamin E family or from a group of structurally or functionally related compounds or derivatives thereof. Examples of said products selected from the vitamin E family or structurally or functionally related compounds or derivatives thereof are listed above in the description of this patent application.

According to yet another embodiment, the present invention relates to an animal feed supplement comprising a suitable amount of vitamin E characterized in that it results in an intake by a ruminant of more than 3 and preferably more than 6 IU vitamin E/kg body weight/day.

Assuming a body weight of 500 kg for a cow, said amount of vitamin E as defined above corresponds to more than 1500 and preferably more than 3000 IU vitamin E/day.

According to yet another embodiment, the present invention relates to an animal feed supplement comprising a suitable amount of vitamin E such that it results in an intake by a ruminant of about 15 to about 20 IU vitamin E/kg body weight/day.

Again, assuming a body weight of 500 kg for a cow, said amount of vitamin E as defined above corresponds to about 7500 IU to about 10 000 IU vitamin E/day. As mentioned before, the inventors investigated a dose of about 9600 IU vitamin E/day effective in increasing the CLA levels. However the effective dose might be lower but above the basal level of 1500 IU/day. A preferred dose of 3000 IU vitamin E/day will prevent substantial reduction in milk fat and consequently it prevents trans-11 to trans-10 shift.

According to a further embodiment, the present invention relates to an animal feed supplement in admixture with a diet increasing the CLA content in milk or tissue fat directly derived from a ruminant. More particularly, said feed supplement can be fed in conjunction with diets that enhance formation of trans11 C_{18:1}, and subsequently CLA content in fat. The composition of such diets are described in published reviews (Griinari and Bauman 1999; Bauman et al. 2000; Chilliard et al. 2000). It is known that vegetable oils or fish oils can be used for said purpose. For example, basal diets consisting of corn grain and/or corn silage supplemented with vegetable or fish oil or products/meals containing these oils can increase CLA content in milk fat. Also, basal diets having insufficient roughage content, i.e. a level of coarse fibre feed in the diet that does not provide for maintenance of rumen environment, can increase CLA content in milk fat. In addition, also diets supplemented with fish oil at levels above 200 g/d for a lactating dairy cow can be used for said purpose. In conjunction of these said diets vitamin E supplementation may be of benefit.

According to a further embodiment the present invention relates to an animal feed.

According to a further embodiment, said animal feed comprises a suitable amount of a chemical compound selected from the group consisting of the vitamin E family. Suitable components are chosen from the list:

1. Derivatives of tocopherols (such as chroman analogs and trolox),

2. Synthetic phenol compounds [such as di-tent-butylhydroxy toluene (BHT), tert-butylhydroxy anisole (BHA)],

3. Natural phenol compounds (such as flavonoids),

4. Secondary antioxidants, such as compounds active in the aqueous phase such as ascorbic acid or compounds active in the lipid phase such as ubiquinol, and derivatives thereof.

Suitable members thereof are vitamin E or compounds of the vitamin E family, comprising α-tocopherol, β-tocopherol, χ-tocopherol, δ-tocopherol, tocotrienols or a structurally or functionally related compound such as derivatives of tocopherols such as chroman analogs and trolox, synthetic phenol compounds, such as di-tert-butylhydroxy toluene (BHT), tert-butylhydroxy anisole (BHA), natural phenol compounds such as flavonoids, secondary antioxidants that support vitamin-E effect such as ascorbic acid or ubiquinol or derivatives or mixtures thereof.

More particularly, said animal feed can alter the trans-11 C_{18:1} fatty acid and CLA content in milk or tissue fat directly derived from a ruminant.

According to a further embodiment, the animal feed comprises an amount of vitamin E of more than 1500, preferably 3000 IU vitamin E/day, with the aim to shift the reaction in the produced milk favouring trans11 C_{18:1} production.

In an example said animal feed may comprise vitamin E in an amount of more than 3000 IU vitamin E/day, more than 4500 IU vitamin E/day, more than, 6000 IU vitamin E/day or more than 7500 IU vitamin E/day.

In a more preferred embodiment, the animal feed comprises more than 5000 IU vitamin E/day.

Several documents are cited throughout this text. Each of the documents cited herein are hereby incorporated by reference, however there is no admission that any document cited is indeed prior art of the present invention.

Further aspects of the present invention will be described in the enclosed non-limiting examples in reference to the following Figures.

### LIST OF FIGURES AND TABLES

FIG. 1.

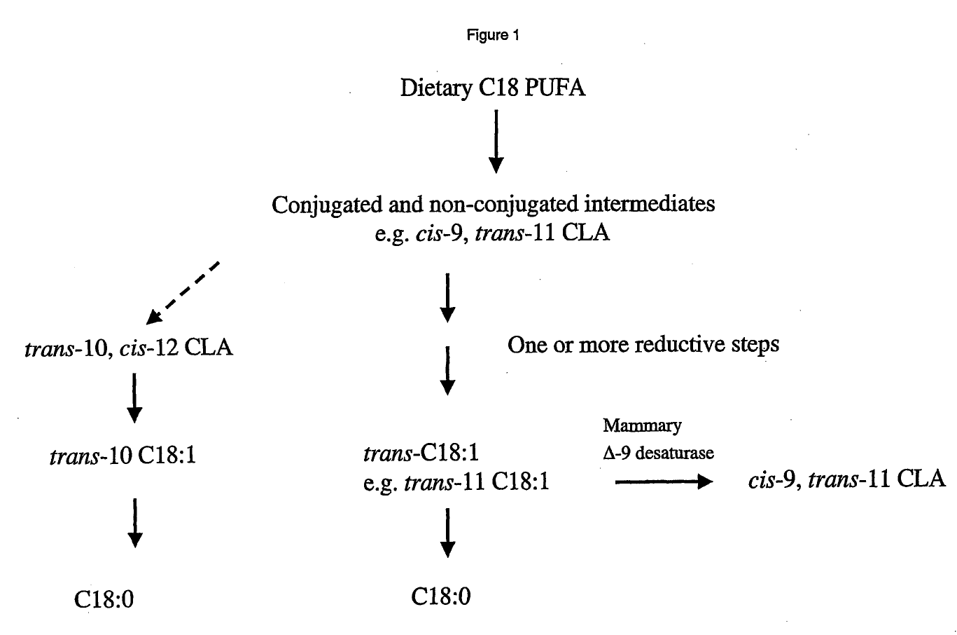

Pathways of ruminal biohydrogenation.

Table 1a.

Effect of dietary fish oil supplementation of dairy cow feeds on rumen digesta outflow of fatty acids (g/d).

Table 1b.

Effect of dietary fish oil supplementation of dairy cow feeds on milk fat composition (% of total fatty acids).

Table 2.

Effect of vitamin E supplementation of dairy cow feeds on milk production.

Table 3.

Effect of vitamin E supplementation of dairy cow diets on milk fat composition and yield of selected fatty acids.

### EXAMPLES

### Example 1

### Effect of Dietary Addition of Fish Oil on Fatty Acid Composition of Milk

The effect of dietary supplementation with fish oil on rumen biohydrogenation, formation of biohydrogenation intermediates in the rumen and subsequent appearance of trans-11 C_{18:1} and CLA in milk fat was tested in a study using 4 lactating dairy cows in late lactation. Control diet was fed during preceding weeks and fish oil supplementation (250 g/d) was carried out during a following 4-week period. Outflow of trans11 C_{18:1} and cis-9, trans-11 CLA formed in the rumen was quantified by using non-digestible markers and frequent sampling of rumen digesta at the omasum.

The inventors have found that when a lactating dairy cow is fed a diet containing 250 g/d fish oil, 100% of the increase in milk fat CLA output is attributable to rumen derived trans-11 C_{18:1} (FIGS. 2a and 2b). This example demonstrates the potential associated with a feeding strategy that enhances ruminal formation of trans-11 C_{18:1} to increase milk fat output of CLA.

### Example 2

### Animal Study to Investigate Dietary Addition of Vitamin E

Experimental Set-Up

The effect of Vitamin E addition in the diet on milk fat depression as well as on the biohydrogenation shift from trans-11 to trans-10 fatty acids classically observed upon feeding of high doses of unsaturated fatty acids, was tested in vivo with six multiparous Holstein cows that yielded more than 25 kg milk/d.

A Latin square experimental design consisting of three groups of two cows, three periods of four weeks and three diets (control, oilseed and oilseed+tocopheryl-acetate) was followed. For all groups, the mixed basal diet consisted, in dry matter terms, of 30% grass silage, 46% corn silage, 13% dried sugar beet pulp, 10% soybean meal and 1% mineral & vitamin mixture. The oilseed concentrate consisted of 50% barley, 25% rapeseed and 25% linseed, whereas its control counterpart was made of 50% barley, 25% rapeseed meal and 25% linseed meal.

The amounts of oilseeds concentrate offered were calculated to provide approximately 550 g/d of supplemental lipids. In addition, cows fed oilseed diet plus Vitamin E received 9600 IU of the vitamin per day, together with the oilseed concentrate.

Milk weights were automatically recorded daily at each milking. Milk samples were taken during the last week of each period and analysed for crude protein, fat and fatty acid profile. Quantification of the trans-C_{18:1} and CLA isomers was performed by gas chromatography as described in Griinari et al. (1998).

Experimental Results

As shown in Table 1, milk yield and protein yield were not significantly affected by the diet. By contrast, the milk fat yield was significantly decreased when oilseeds were included in the diet. Vitamin E supplementation resulted in a complete recovery of milk fat yield.

Table 2 shows that the addition of oilseeds in the diet had a dramatic effect on the milk fatty acid profile: significantly less C_{4:0} to C_{16:0} saturated fatty acids but higher levels of stearic acid (C_{18:0}); significantly more total monounsaturated C₁₈ fatty acids, significantly higher levels of the major trans and conjugated fatty acids (trans-10 C_{18:1}, trans-11 C_{18:1}; cis-9, trans-11 C_{18:2}).

The supplementation of the oilseed diet with Vitamin E had no major impact on the levels of C_{4:0} to C_{16:0} saturated fatty acids, stearic acid and total C_{18:1} fatty acids. In contrast, vitamin E altered the trans fatty acids profile and increased CLA content dramatically. The level of trans-10 C_{18:1} decreased to control levels and the level of trans-11 C18:1 increased significantly. The effect of vitamin E on trans-10 C_{18:1} and CLA was even more pronounced in terms of yields.

Interpretation of Experimental Results

In terms of trans fatty acids, the values obtained in the milk clearly indicate that the oilseed diet induces a shift in the ruminal biohydrogenation process, allowing a proportionally higher amount of trans-10 C_{18:1} than of trans-11 C_{18:1} to be formed. This previously described shift in major biohydrogenation intermediates (Griinari and Bauman, 1999) was completely abolished by the supplementation of the oilseed diet with vitamin E. This vitamin E effect allows the rumen to produce more trans-11 C_{18:1}, which explains the increased content of trans-11 C_{18:1} in milk fat.

Finally, the present invention clearly shows a significant increase of the most important milk fat CLA (cis-9, trans-11 C_{18:2}) upon feeding an oilseed rich diet supplemented with a high dose of vitamin E. The effect was significant in terms of fatty acid profiles, but it was even more impressive in terms of yields. Average daily yields of CLA were 4.9 g, 9.0 g and 15.6 g from cows fed control, oilseed and oilseed+vitamin E diets, respectively.

### REFERENCES

1; Adlof R. O., Duval S., Emken E. A. 2000. Biosynthesis of conjugated linoleic acid in humans. Lipids 35:131-135.

2. Aro A, Männistö S, Salminen I, Ovaskainen M- L, Kataja V, Uusitupa M. 2000. Inverse association between dietary and serum conjugated linoleic acid and risk of breast cancer in postmenopausal women. Nutrition and Cancer 38:151-157.

3. Bauman, D. E., Griinari, J. M., 2000. Regulation and nutritional manipulation of milk fat: low-fat milk syndrome. In: Mol, J. A., Clegg, R. A. (Eds.), Biology of the Mammary Gland. Kluwer Academic/Plenum Publishers, New York, pp. 209-216.

4. Bauman, D. E., Barbano, D. M., Dwyer, D. A., Griinari, J. M., 2000a. Technical Note: Production of butter with enhanced conjugated linoleic acid for use in biomedical studies with animal models. J. Dairy Sci. (in press) (Correction ???).

5. Bauman, D. E., Baumgard, L. H., Corl, B. A., Griinari, J. M., 2000b. Biosynthesis of conjugated linoleic acid in ruminants. Proc. Am. Soc. Anim. Sci. 1999. Available at: http//www.asas.org/jas/symposia/proceedings/0937.pdf.

6. Baumgard, L. H., B. A. Corl, D. A. Dwyer, A. SΦbρ, and D. E. Bauman. 2000. Identification of the conjugated linoleic acid isomer that inhibits milk fat synthesis. Am. J. Physiol. 278:R179-R184.

7. Charmley, E., J. W. G. Nicholson, and J. A. Zee. 1993. Effect of supplemental vitamin E and selenium in the diet on vitamin E and selenium levels and control of oxidized flavor in milk from Holstein cows. Can. J. Anim. Sol. 73:453-457.

8. Chilliard, Y., A. Ferlay, R. M. Mansbridge, and M. Doreau. 2000. Ruminant milk fat plasticity: nutritional control of saturated, polyunsaturated, trans and conjugated fatty acids. Ann. Zootech. 49:181-205.

9. Fritsche, J., R. Rickert, H. Steinhart, M. P. Yurawecz, J. A. G. Roach, J. K. G. Kramer, ja Y. Ku. 1999. Conjugated linoleic acid (CLA) isomers: formation, analysis, amounts in foods and dietary intake. Fett/Lipid 101:272-276.

10. Griinari, J. M., B. A. Corl, S. H. Lacy, P. Y. Chouinard, K. V. V. Nurmela, and D. E. Bauman. 2000. Conjugated linoleic acid is synthesized endogenously in lactating dairy cows by 0-9 desaturase. J. Nutr. 130:2285-2291.

11. Griinari, J. M., and D. E. Bauman. 1999. Biosynthesis of conjugated linoleic acid and its incorporation into meat and milk in ruminants. Pages 180-200 in Advances in Conjugated Linoleic Acid Research, Volume 1. M. P. Yurawecz, M. M. Mossoba, J. K. G. Kramer, M. W. Pariza, and G. J. Nelson, eds. AOCS Press, Champaign, Ill.

12.Griinari, J. M., K. Nurmela, D. A. Dwyer, D. M. Barbano, and D. E. Bauman. 1999. Variation of milk fat concentration of conjugated linoleic acid and milk fat percentage is associated with a change in ruminal biohydrogenation. J. Anim. Sci. 77(Suppl. 1):117-118 (Abstr.).

13.Griinari, J. M., D. A. Dwyer, M. A. McGuire, D. E. Bauman, D. L. Palmquist, and K. V. V. Nurmela. 1998. Trans-octadecenoic acids and milk fat depression in lactating dairy cows. J. Dairy Sci. 81:1251-1261.

14. Hodgson, J. M., Wahlqvist M. L., Boxall J. A., Balazs N. D. 1996. Platelet trans fatty acids in relation to angiographically assessed coronary artery disease. Atherosclerosis 120:147-154.

15. INRA, 1988. Alimentation des bovines, ovins & caprins. Jarrige, R. (Ed.), Paris, France.

16. Ip, C., S. Banni, E. Angioni, G. Carta, J. McGinley, H. J. Thompson, D. Barbano, and D. Bauman. 1999. Conjugated linoleic acid-enriched butter alters mammary gland morphogenesis and reduces cancer risk in rats. J. Nutr. 129:2135-2142.

17. Ip, C., Lisk, D. J., Scimeca, J. A. 1994 Potential food modification in cancer prevention. Cancer Res. 54(7):1957s-1959s.

18. Knekt, P., R. Järvinen, R. Seppänen, E. Pukkala, and A. Aromaa. 1996. Intake of dairy products and the risk of breast cancer. Brit. J. Cancer 73:687-691.

19. Reaney, M. J. T., Y. -D. Liu, and N. D. Westcott. 1999. Pages 39-54 in Advances in Conjugated Linoleic Acid Research, Volume 1. M. P. Yurawecz, M. M. Mossoba, J. K. G. Kramer, M. W. Pariza, and G. J. Nelson, eds. AOCS Press, Champaign, Ill.

20. Palmquist, D. L. 1984. Use of fats in diets for lactating dairy cows. In Fats in animal nutrition. J. Wiseman (Ed.), Butterworths, p. 357-381.

21. Pariza, M. W., and W. A. Hargraves. 1985. A beef-derived mutagenesis modulator inhibits initiation of mouse epidermal tumors by 7,12-dimethylbenz[a]anthracene. Carcinogenesis 6:591-593.

22. Pariza, M. W. 1999. the biological activities of conjugated linoleic acid. Pages 12-20 in Advances in Conjugated Linoleic Acid Research, Volume 1. M. P. Yurawecz, M. M. Mossoba, J. K. G. Kramer, M. W. Pariza, and G. J. Nelson, eds. AOCS Press, Champaign, Ill.

23. Piperova, L. J. Sampugna, B. Teter, K. Kalscheur, and R. Erdman. 2000. Comparison of trans octadecenoic isomer profiles in duodenal and milk lipids of cows fed different diets. J. Dairy Sci. Suppl.1 83:277 (Abst.).

24. Sehat, N., R. Rickert, M. M. Mossoba, J. K. G. Kramer, M. P. Yurawezc, J. A. G. Roach, R. O. Adlof, K. M. Morehouse, J. Fritsche, K. D. Eulitz, H. Steinhart, and Y. Ku. 1999. Improved separation of conjugated fatty acid methyl esters by silver ion high-performance lipid chromatography. Lipids 34:407-413.

25. Smith, K. L., J. H. Harrison, D. D. Hancock, D. A. Todhunter, and H. R. Conrad. 1984. Effect of vitamin E and selenium supplementation on incidence of clinical mastitis and duration of clinical symptoms. J. Dairy Sci. 67:1293-1300.

1. Method for increasing the content of trans-11 C_{18:1} fatty acid and CLA in milk or tissue fat directly derived from a ruminant, comprising the step of administering to said ruminant a suitable amount of a chemical compound selected from the group consisting of the vitamin E family, comprising α-tocopherol, β-tocopherol, χ-tocopherol, δ-tocopherol, tocotrienols or a structurally or functionally related compound such as derivatives of tocopherols such as chroman analogs and trolox, synthetic phenol compounds, such as di-tett-butylhydroxy toluene (BHT), tert-butylhydroxy anisole (BHA), natural phenol compounds such as flavonoids, secondary antioxidants that support vitamin-E effect such as ascorbic acid or ubiquinol or derivatives or mixtures thereof.

2. A method according to claim 1 wherein said chemical compound is a member of the vitamin E family, more preferably all-rac-α-tocopheryl acetate.

3. A method according to claim 2 wherein said suitable amount results in administering more than 3 IU, preferably more than 6 IU vitamin E/kg body weight/day.

4. A method according to claims 2 and 3 wherein said suitable amount results in administering about 15 to about 20 IU vitamin E/kg body weight/day.

5. A method according to any of claims 1 wherein said chemical compound of the vitamin E family or structurally or functionally related compound or derivative thereof is administered in conjunction with a diet increasing the CLA content in milk or tissue fat.

6. A method according to claim any of claims 1-5 wherein said ruminant is a lactating ruminant.

7. A method according to claim 6 wherein said lactating ruminant is a cow.

8. A method according to any of claims 1-7 for increasing the CLA content in milk or tissue fat directly derived from a ruminant.

9. A method according to any of claims 1-8 for increasing the trans-11 C_{18:1} fatty acid content in milk or tissue fat directly derived from a ruminant.

10. A method according to any of claims 1-9 for increasing the content of both trans-11 C_{18:1} fatty acid and CLA, without simultaneously increasing the trans 10 C_{18:1} fatty acid content in milk or tissue fat directly derived from a ruminant.

11. A method according to any of claims 1-10 for reducing the formation of trans-10 C_{18:1} fatty acid in the rumen.

12. Raw milk or tissue fat obtainable by the method of any of claims 1-11.

13. Raw milk or tissue fat according to claim 12 having a fatty acid composition comprising at least 1% of the total fatty acids in the form of cis-9, trans-11 C_{18:2}.

14. Raw milk or tissue fat according to claims 12 or 13 having a fatty acid composition comprising at least 2% of the total fatty acids in the form of trans-11 C_{18:1}.

15. Raw milk or tissue fat according to any of claims 12-14 having a fatty acid composition characterized by a trans-11 C_{18:1} trans-10 C_{18:1} ratio higher than 2.

16. A nutritional product comprising the milk or tissue fat according to any of claims 12-15 or a derivative thereof.

17. An animal feed supplement suitable for use in the method according to any of claims 1-11.

18. An animal feed supplement comprising a suitable amount of vitamin E family member, characterized in that it results in an intake by a ruminant of more than 3 and preferably more than 6 IU vitamin E/kg body weight/day.

19. An animal feed supplement according to claim 17 or 18 in admixture with a diet increasing the CLA content in milk or tissue fat directly derived from a ruminant.

20. An animal feed comprising a feed supplement according to any of claim 18-19.

21. An animal feed able to alter the trans-11 C_{18:1} fatty acid and CLA content in milk or tissue fat directly derived from a ruminant, comprising a suitable amount of a chemical compound selected from the group consisting of the vitamin E family, comprising α-tocopherol, β-tocopherol, χ-tocopherol, δ-tocopherol, tocotrienols or a structurally or functionally related compound such as derivatives of tocopherols such as chroman analogs and trolox, synthetic phenol compounds, such as di-tert-butylhydroxy toluene (BHT), tert-butylhydroxy anisole (BHA), natural phenol compounds such as flavonoids, secondary antioxidants that support vitamin-E effect such as ascorbic acid or ubiquinol or derivatives or mixtures thereof.

22. An animal feed according to claim 20-21 comprising vitamin E in an amount of more than 1500, preferably more than 3000 IU vitamin E/day.

23. An animal feed according to claim 22 comprising vitamin E in an amount of more that at least 5000 IU vitamin E/day.
